(19) United States
(12) Patent Application Publication
(10) Pub. No.: US 2017/0028225 A1
(43) Pub. Date: Feb. 2, 2017

(54) ULTRASONIC PROBE AND ULTRASONIC APPARATUS

(71) Applicant: Seiko Epson Corporation, Tokyo (JP)

(72) Inventor: Robina ATSUCHI, Fujimi (JP)

(21) Appl. No.: 15/210,050

(22) Filed: Jul. 14, 2016

(30) Foreign Application Priority Data
Jul. 30, 2015 (JP) 2015-150419

(51) Int. Cl. A61N 7/00

(52) U.S. Cl. CPC A61N 7/00; A61N 2007/0078; A61N 2007/0073

(57) ABSTRACT

An ultrasonic probe includes a flexibly longitudinal first sheet portion and a plurality of flexible second sheet portions that are connected to one surface side of the first sheet portion. Each second sheet portion has a connection surface to which the first sheet portion is connected and a drive surface on a side opposite to the connection surface and includes a plurality of ultrasonic substrates respectively including ultrasonic transducers each of which can output an ultrasonic wave from the drive surface.

### BACKGROUND

1. Technical Field

The present invention relates to an ultrasonic probe and an ultrasonic apparatus.

2. Related Art

In the related art, there is a known ultrasonic apparatus which performs treatment of a predetermined site of a living body and performs improvement of constitution by using an ultrasonic wave (for example, refer to JP-A-2013-000348).

JP-A-2013-000348 discloses health equipment in which an ultrasonic oscillation element, a heater, and a coolant are provided on a piece of flexible cloth. The health equipment can perform treatment while switching between hyperthermic treatment and cold treatment. During the hyperthermic treatment, the heater and an ultrasonic oscillation device are driven. During the cold treatment, the coolant and the ultrasonic oscillation element are driven.

Incidentally, in JP-A-2013-000348, a gel layer is provided on a piece of cloth and the gel layer is brought into close contact with a desired site of a living body so that the living body and health equipment are brought into close contact with each other. However, in such a configuration, there are cases in which it is difficult to appropriately bring the living body and the health equipment into close contact with each other, and the propagation efficiency of an ultrasonic wave with respect to the living body is deteriorated. For example, in JP-A-2013-000348, the health equipment is brought into close contact with a treatment site so as to cover the treatment site in its entirety. However, as the size of the treatment site increases, air bubbles are likely to enter a space between a living body and the health equipment, and the propagation efficiency of an ultrasonic wave is deteriorated.

In a case where treatment or the like is performed with respect to a living body by using an ultrasonic wave, there is a need to change the output of the ultrasonic wave depending on the site or the symptom of the living body. In JP-A-2013-000348 described above, there is a problem in that an appropriate ultrasonic wave with respect to each of the sites of a living body cannot be output.

### SUMMARY

An advantage of some aspects of the invention is to provide an ultrasonic probe which can come into close contact with a living body and can transmit an ultrasonic wave in an optimum output condition, and an ultrasonic apparatus.

An ultrasonic probe according to an application example of the invention includes: a flexibly longitudinal first sheet portion; and a plurality of flexible second sheet portions that are connected to one surface side of the first sheet portion. Each second sheet portion has a connection surface to which the first sheet portion is connected and a drive surface on a side opposite to the connection surface and includes a plurality of ultrasonic transducers each of which can output an ultrasonic wave from the drive surface.

In the ultrasonic probe according to the application example, the plurality of second sheet portions are provided with respect to the first sheet portion, and the plurality of ultrasonic transducers are provided on the drive surface of each second sheet portion. In such a configuration, the drive surfaces of the second sheet portions can adhere to a site of a living body required to be treated while preventing an air layer from being interposed therebetween. Incidentally, the curvature of a skin surface of a living body varies depending on a site or due to individual differences. Therefore, health equipment in the related art disclosed in JP-A-2013-000348 cannot adhere to such a shape of a living body in a state where an appropriate ultrasonic wave can be output. In contrast, in the application example, both the first sheet portion and the second sheet portions are formed to have flexibly longitudinal shapes. In this case, the second sheet portions can be disposed such that the air layer does not enter a space between a living body and the second sheet portions by causing longitudinal directions of the second sheet portions to follow along a curved surface of the living body.

Accordingly, the ultrasonic probe can come into close contact with a living body, and the ultrasonic wave is not reflected or attenuated by the air layer. Thus, it is possible to output an ultrasonic wave in optimum output conditions.

In the ultrasonic probe according to the application example, it is preferable that the second sheet portions are provided so as to be line-symmetrical with respect to the first sheet portion.

In the application example with this configuration, the second sheet portions are provided so as to be line-symmetrical while having the first sheet portion as the center.

Generally, a living body such as a human being, for example, has a structure bilaterally symmetrical with respect to the central axis. For example, in a case of a torso, the back, the chest, and the shoulders are shaped so as to have substantially bilateral symmetry while having the backbone as the center. In a case of a face, while having the ridge of the nose as the center, the eyes, the mouth, and the cheeks are shaped so as to have substantially bilateral symmetry.

In a case where the ultrasonic probe is attached to such a living body, in the application example, the longitudinal direction of the first sheet portion can be aligned with the central axis of the living body, and the second sheet portions which are line-symmetrical with respect to the first sheet portion can be disposed at sites where the second sheet portions respectively become line-symmetrical with respect to the central axis of the living body at equal distances. For example, in a case where the ultrasonic probe is used in improvement of lumbago, the first sheet portion is disposed along the backbone. Then, it is possible to easily dispose one side of the second sheet portions at the left side waist and the other side of the second sheet portions at the right side waist. Accordingly, when the ultrasonic probe adheres to a living body, the second sheet portions may be caused to adhere thereto after the first sheet portion is positioned along the central axis of the living body. Thus, the ultrasonic probe is easily fixed to the living body.

In the ultrasonic probe according to the application example, it is preferable that the second sheet portions are longitudinal in a direction intersecting a longitudinal direction of the first sheet portion.

In the application example with this configuration, the second sheet portions are configured to have shapes longitudinal in the direction intersecting the longitudinal direction of the first sheet portion. Accordingly, as described above, after the first sheet portion is disposed on the central axis of a living body (for example, a site corresponding to the backbone), the second sheet portions are bilaterally spread, thereby being able to be widely disposed in a desired area of the living body.

In the ultrasonic probe according to the application example, it is preferable that each second sheet portion has a longitudinal direction and each second sheet portion is provided with wiring which is connected to the ultrasonic transducers and is meandering in the longitudinal direction of the second sheet portion.

In the application example with this configuration, each second sheet portion is provided with the wiring meandering in the longitudinal direction. As described above, since the second sheet portion is flexible, when the second sheet portion is curved along the shape of a living body or the second sheet portions are pulled, tensile stress is applied to the wiring. Here, for example, in a case where the wiring connected to each of the ultrasonic transducers is linear and parallel to the longitudinal direction, there is a possibility of disconnection due to tensile stress applied to the wiring.

In contrast, the wiring in the application example is meandering in the longitudinal direction of the second sheet portion. Therefore, even in a case where the second sheet portion is caused to follow or extend along the curved shape, the wiring is stretched from a meandering state to a linear state. Thus, it is possible to release the stress and to reduce the risk of disconnection.

In the ultrasonic probe according to the application example, it is preferable that each second sheet portion has wiring which is connected to the ultrasonic transducers and the wiring connects the plurality of ultrasonic transducers together in parallel.

In the application example with this configuration, the plurality of ultrasonic transducers provided in each second sheet portion are connected together in parallel through the wiring. In such a configuration, the same voltage can be applied to each of the ultrasonic transducers connected together in parallel, and ultrasonic waves can be output from the ultrasonic transducers at the same time. Particularly, in a case where ultrasonic treatment is performed by outputting an ultrasonic wave from the ultrasonic probe to a living body, there is no need to receive an ultrasonic wave. As described above, it is possible to simplify the structure of the wiring by providing a configuration specialized for transmitting an ultrasonic wave.

In the ultrasonic probe according to the application example, it is preferable that each ultrasonic transducer includes a first ultrasonic transducer which can output an ultrasonic wave having a first frequency and a second ultrasonic transducer which can output an ultrasonic wave having a second frequency different from the first frequency.

In the application example with this configuration, each ultrasonic transducer disposed in the plurality of second sheet portions includes the first ultrasonic transducer which can output an ultrasonic wave having the first frequency and the second ultrasonic transducer which can output an ultrasonic wave having the second frequency. In such a configuration, in accordance with a site to which an ultrasonic wave is transmitted by the ultrasonic probe, the ultrasonic transducers to output an ultrasonic wave can be switched. Accordingly, an ultrasonic wave can be output in accordance with a site which is the output destination of the ultrasonic wave, and for example, it is possible to avoid the risk of destruction or the like of biological tissue caused by an ultrasonic wave. In a case where an ultrasonic wave is intended to be sent to a shallow portion inside a living body, an ultrasonic wave having a high frequency is used. In a case where an ultrasonic wave is intended to be sent to a deep portion, an ultrasonic wave having a low frequency is used. In this manner, the transmission depth of an ultrasonic wave can also be changed by changing the to-be-driven ultrasonic transducer.

In the ultrasonic probe according to the application example, it is preferable that the second sheet portions are provided so as to be attachable/detachable with respect to the first sheet portion.

In the application example with this configuration, the second sheet portions are provided so as to be attachable/detachable with respect to the first sheet portion.

Accordingly, for example, in a case where a portion among the plurality of second sheet portions is unnecessary, the portion can be easily removed from the first sheet portion. Therefore, a user does not feel discomfortable due to the unnecessary second sheet portion. Since the second sheet portions are attachable/detachable with respect to the first sheet portion, even when the ultrasonic probe is removed from a living body, it is possible to perform an operation in which the second sheet portions are removed piece by piece or the like. Thus, it is possible to improve the operability.

In the ultrasonic probe according to the application example, it is preferable that the first sheet portion includes first magnets at positions where the first sheet portion is connected to the second sheet portions and each second sheet portion includes a second magnet having magnetism opposite to that of the first magnet and is connected to the first sheet portion due to magnetic force.

In the application example with this configuration, the first sheet portion and the second sheet portions are connected to each other by the magnet. Accordingly, when the second sheet portions are torn off in a direction of being separated from the first sheet portion, the first sheet portion and the second sheet portions can be easily separated from each other. The second sheet portions can be easily connected to the first sheet portion by only causing the second sheet portions to approach the first sheet portion. In this case, particularly, the configuration is advantageous when the ultrasonic probe is attached and detached with respect to a position which is out of view (the back or the like) of a user.

In the ultrasonic probe according to the application example, it is preferable that the first sheet portion has power transmitting coils at positions where the first sheet portion is connected to the second sheet portions and each second sheet portion has a power receiving coil at a position where the second sheet portion is connected to the first sheet portion, and power is supplied from the power transmitting coils to each power receiving coil through contactless power transmission.

In the application example with this configuration, power is supplied from the power transmitting coils provided in the first sheet portion to each power receiving coil of the second sheet portion through contactless power transmission. Accordingly, even though the first sheet portion and the second sheet portions are members different from each other, it is possible to supply power from the first sheet portion to each second sheet portion provided with the ultrasonic transducers.

An ultrasonic apparatus according to another application example of the invention includes the ultrasonic probe described above and a control unit that controls the ultrasonic probe.

In the ultrasonic apparatus according to the application example, as described above, the ultrasonic probe can be brought into close contact with and be attached to a living body such that an appropriate ultrasonic wave can be output. Accordingly, it is possible to enhance the effect of ultrasonic treatment by causing the control unit to control the ultrasonic probe and outputting an ultrasonic wave to the inside of a living body.

In the ultrasonic apparatus according to the application example, it is preferable to include a storage unit that stores a target subject which is an output target of an ultrasonic wave and sequential data which indicates an output procedure of an ultrasonic wave with respect to the target subject. It is preferable that the control unit causes a plurality of ultrasonic transducers to respectively output ultrasonic waves, based on the sequential data.

In the application example with this configuration, the sequential data is stored in the storage unit, and the control unit causes the ultrasonic probe to output an ultrasonic wave based on the sequential data corresponding to the target subject (tissue inside a living body) which becomes the output target of the ultrasonic wave. Accordingly, it is possible to perform optimum ultrasonic treatment corresponding to the output target of an ultrasonic wave.

In the ultrasonic apparatus according to the application example, it is preferable that the sequential data includes drive element data which indicates the position of a to-be-driven ultrasonic transducer among the plurality of ultrasonic transducers and the control unit selects the to-be-driven ultrasonic transducer from the plurality of ultrasonic transducers based on the drive element data and drives the selected ultrasonic transducer.

In the application example with this configuration, the control unit selects the to-be-driven ultrasonic transducer based on the drive element data included in the sequential data. In other words, depending on the biological tissue, there are cases where a risk of destruction in biological tissue is present due to excessively strong intensity of an ultrasonic wave, and there are cases where a sufficient treatment effect cannot be obtained due to excessively weak intensity of an ultrasonic wave. In the application example, the to-be-driven ultrasonic transducer is selected and the selected ultrasonic transducer is driven in accordance with the target subject which is the output destination of an ultrasonic wave. In this case, for example, the number of the to-be-driven ultrasonic transducers can be increased or decreased, or an ultrasonic transducer which can output an ultrasonic wave having a frequency corresponding to the depth of the target subject can be selected. Accordingly, it is possible to output an ultrasonic wave having optimum intensity or frequency corresponding to the target subject.

In the ultrasonic apparatus according to the application example, it is preferable that the sequential data includes output range data which indicates an output range of an ultrasonic wave and the control unit performs delay-driving of the plurality of ultrasonic transducers based on the output range data.

The output range of an optimum ultrasonic wave with respect to biological tissue is determined in advance. For example, in comparison between an arm and a leg, since the leg is thicker than the other portion, there is a need to output an ultrasonic wave in a wider range. In contrast, in the application example with this configuration, the sequential data includes the output range data, and the control unit performs delay-driving of the plurality of ultrasonic transducers based on the output range data. In other words, the inclinations of wave surfaces of ultrasonic waves output from the plurality of ultrasonic transducers can be controlled by gradually delaying driving of the plurality of ultrasonic transducers, and the ultrasonic waves can be output in a predetermined direction. Accordingly, it is possible to output an ultrasonic wave in an optimum output range in accordance with the target subject by performing scanning control of an emission direction of the ultrasonic wave such that the ultrasonic wave is output within a range based on the output range data.

### BRIEF DESCRIPTION OF THE DRAWINGS

The invention will be described with reference to the accompanying drawings, wherein like numbers reference like elements.

FIG. 1 is a view illustrating a schematic configuration of an ultrasonic apparatus according to a first embodiment of the invention.

FIG. 2 is a plan view illustrating a schematic configuration of an ultrasonic probe of the present embodiment when viewed from a side of an adhesive surface (a drive surface) with respect to a living body.

FIG. 3 is a schematic cross-sectional view of a second sheet portion of the present embodiment, taken along line A-A in FIG. 2.

FIG. 4 is a plan view illustrating a schematic configuration of an element substrate of the present embodiment.

FIG. 5 is a sectional view of an element substrate, a sealing plate, and a wiring substrate of the present embodiment, taken along line B-B in FIG. 4.

FIG. 6 is a view illustrating a fixed position in a case where the ultrasonic probe is adherently applied to the back, according to the present embodiment.

FIG. 7 is a view illustrating a fixed position in a case where the ultrasonic probe is adherently applied to the heel, according to the present embodiment.

FIG. 8 is a flow chart illustrating a treatment method of a treatment site performed by using the ultrasonic apparatus, according to the present embodiment.

FIG. 9 is a schematic plan view of an ultrasonic probe according to a second embodiment of the invention when viewed from a side of an adhesive surface with respect to a living body.

FIGS. 10A and 10B are plan views respectively illustrating a first sheet portion and a second sheet portion of an ultrasonic probe in the vicinity of a connection portion therebetween, according to a third embodiment of the invention.

FIG. 11 is a perspective view illustrating a schematic configuration of an ultrasonic probe, according to a fourth embodiment of the invention.

FIG. 12 is a plan view illustrating a schematic configuration of the ultrasonic probe, according to the fourth embodiment.

FIG. 13 is a plan view illustrating another schematic configuration of the ultrasonic probe, according to the fourth embodiment.

FIG. 14 is a perspective view illustrating a schematic configuration of an ultrasonic probe, according to a modification example.

### DESCRIPTION OF EXEMPLARY EMBODIMENTS

### First Embodiment

Hereinafter, a first embodiment of the invention will be described with reference to the drawings.

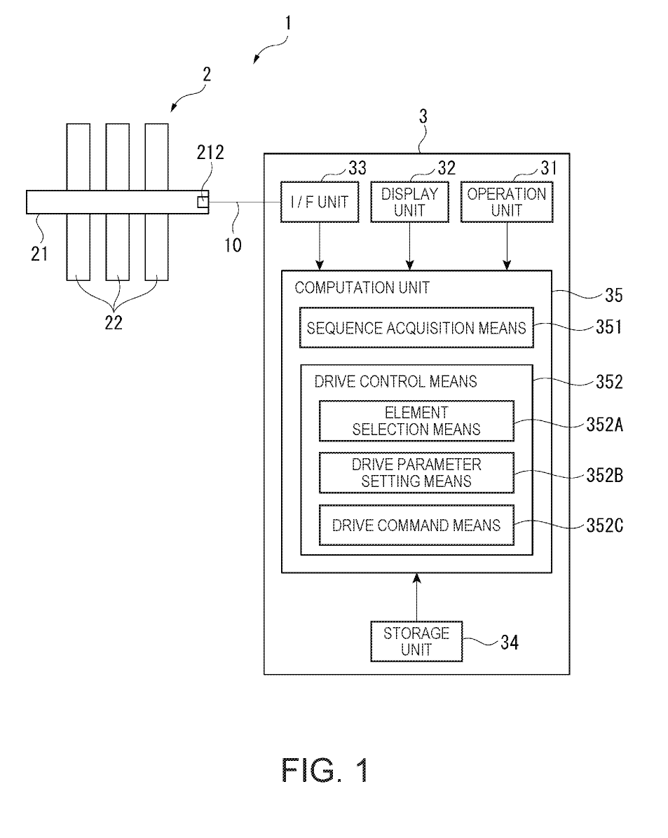

FIG. 1 is a view illustrating a schematic configuration of an ultrasonic apparatus 1, according to the first embodiment.

As illustrated in FIG. 1, the ultrasonic apparatus 1 according to the present embodiment includes an ultrasonic probe 2 and a control device 3 which controls the ultrasonic probe. The ultrasonic probe 2 and the control device 3 are connected to each other through a cable 10.

In the ultrasonic apparatus 1, ultrasonic treatment is performed by adherently applying the ultrasonic probe 2 to a treatment site of a living body and outputting an ultrasonic wave from the ultrasonic probe 2 to the inside of the living body through the control of the control device 3. For example, such ultrasonic treatment includes improvement of shoulder stiffness, lumbago, and arthralgia; fat combustion assistance performed by activating visceral fat; skin care performed by activating a skin surface; and massage of muscular tissue of a living body. In other words, “the ultrasonic treatment” mentioned in the invention is not limited to treatment for a discomfort portion of a living body performed by using an ultrasonic wave. The ultrasonic treatment also includes additional improvement of a bodily function and application of a relaxing effect. In the subsequent description, a site of a living body in which the aforementioned ultrasonic treatment is performed will be referred to as the treatment site, and contents of the ultrasonic treatment will be referred to as contents of treatment.

Hereinafter, each portion in a configuration of the ultrasonic apparatus 1 will be described in detail.

### Configuration of Ultrasonic Probe 2

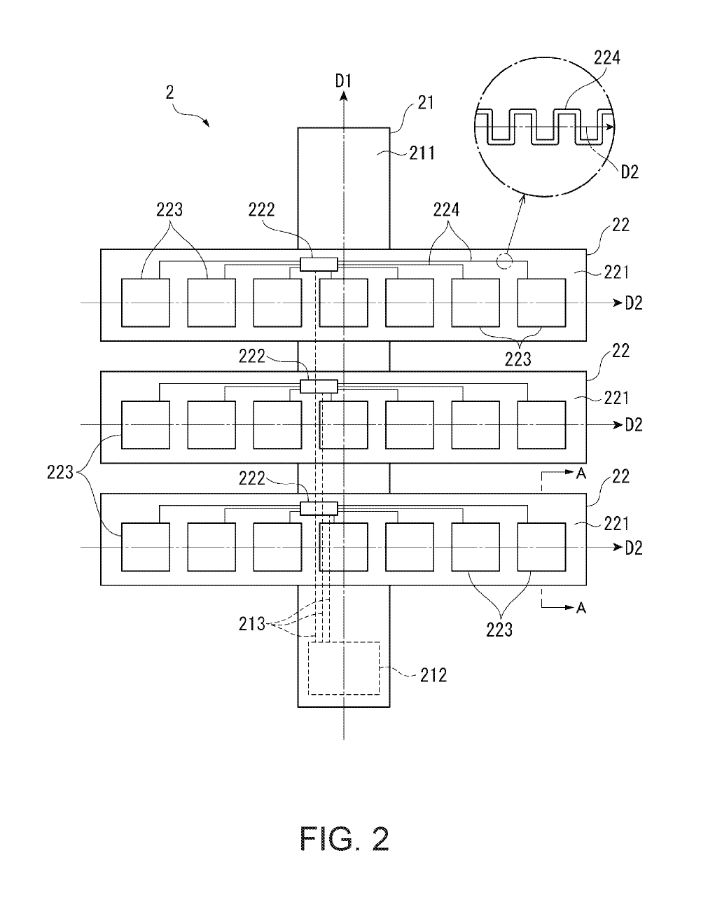

FIG. 2 is a plan view illustrating a schematic configuration of the ultrasonic probe 2 of the present embodiment when viewed from a side of an adhesive surface (drive surface) with respect to a living body.

As illustrated in FIGS. 1 and 2, the ultrasonic probe 2 includes a longitudinal first sheet portion 21 and second sheet portions 22 which are longitudinal in a direction intersecting a longitudinal direction of the first sheet portion 21.

### Configuration of First Sheet Portion 21

For example, the first sheet portion 21 includes a first sheet main body portion 211 which is configured to be made from a soft synthetic resin such as a vinyl chloride resin.

A connector portion 212 is provided on one end side of the first sheet main body portion 211 in the longitudinal direction. The cable 10 for communicating with the control device 3 is connected to the connector portion 212 in an attachable/detachable manner.

Inside the first sheet main body portion 211, there are provided a plurality of connection wiring portions 213 connected to the connector portion 212. The connection wiring portions 213 are provided so as to respectively correspond to the second sheet portions 22. The connection wiring portions 213 are provided along the longitudinal direction of the first sheet portion 21 (D1 direction in FIG. 2) and a tip portion of each connection wiring portion 213 is connected to a branch circuit portion 222 provided in each second sheet portion 22.

### Configuration of Second Sheet Portion 22

The second sheet portions 22 are longitudinally formed in a direction orthogonal (D2 direction) to the longitudinal direction of the first sheet portion 21 (D1 direction). The second sheet portions 22 are bonded to the first sheet portion 21 at central portions thereof in the longitudinal direction. In other words, in the present embodiment, the second sheet portions 22 have shapes line-symmetrical while having the first sheet portion 21 as the center.

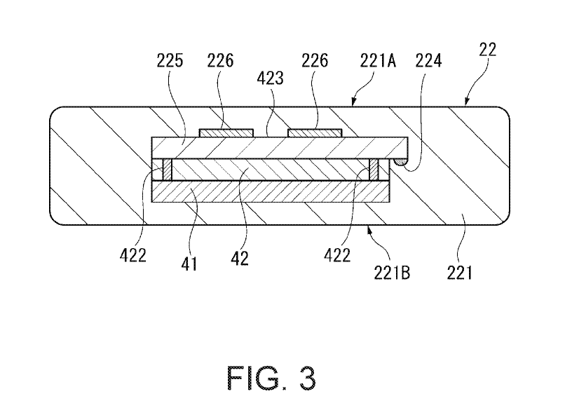

For example, each second sheet portion 22 is configured to be made from synthetic rubber and includes a flexible and elastic second sheet main body portion 221. The second sheet main body portion 221 has a connection surface 221A on the first sheet portion 21 side (refer to FIG. 3) and a drive surface 221B on a side opposite to the connection surface 221A (refer to FIG. 3). At the central position of the second sheet main body portion 221 in the longitudinal direction, the connection surface 221A is glued and bonded to the first sheet main body portion 211 by using an adhesive or performing thermocompression bonding, for example.

The present embodiment illustrates an example in which the first sheet portion 21 and the second sheet portions 22 are bonded to each other on the connection surface 221A. However, the embodiment is not limited thereto. For example, an attachable/detachable configuration may be adopted. An example of the attachable/detachable configuration will be described in the below-described third embodiment.

The second sheet main body portion 221 has a configuration in which the drive surface 221B can adhere to a living body. Specifically, the drive surface 221B of the second sheet main body portion 221 is provided with a fixing layer which can come into close contact with a living body and can be fixed to the living body. For example, such a fixing layer may be a gel-like fixing layer which is applied to the drive surface 221B when the ultrasonic probe 2 is fixed to a living body, or may be a sticky sheet which is adherently applied to the drive surface 221B.

As illustrated in FIG. 2, the branch circuit portions 222 and a plurality of ultrasonic substrates 223 are provided inside the second sheet main body portion 221.

As described above, each connection wiring portion 213 provided in the first sheet portion 21 is connected to each branch circuit portion 222. Specifically, at a connection portion of the first sheet portion 21 and the second sheet portions 22, the connection wiring portions 213 penetrates the insides of the second sheet main body portions 221 in addition to the inside of the first sheet main body portion 211, thereby being respectively connected to the branch circuit portions 222. Inside each second sheet main body portion 221, signal lines 224 (wiring) are respectively wired to the ultrasonic substrates 223 from the branch circuit portion 222. Accordingly, power or drive command data supplied from the control device 3 is transmitted to the ultrasonic substrates 223 in each second sheet portion 22.

As illustrated in the enlarged view at the upper right of FIG. 2, each signal line 224 is disposed meandering in the longitudinal direction of the second sheet portion 22 (D2 direction in FIG. 2). Accordingly, for example, even in a case where the second sheet main body portion 221 extends along the D2 direction, the signal line 224 is deformed so as to be linear and parallel to the D2 direction. Therefore, tensile force applied to the signal line 224 can be released, and the risk of disconnection can be prevented. FIG. 2 exemplifies an example in which the single-lined signal line 224 is connected to each ultrasonic substrate 223. However, multiple signal lines 224 may be connected to each ultrasonic substrate 223.

### Configuration of Ultrasonic Substrate 223

The plurality of ultrasonic substrates 223 are provided inside each second sheet main body portion 221 along the longitudinal direction of the second sheet portion 22 (D2 direction). In order to simplify the description, FIG. 2 illustrates an example in which seven ultrasonic substrates 223 are provided in one second sheet portion 22. However, actually, more ultrasonic substrates 223 are provided while having narrower gaps thereamong. Although a configuration in which the ultrasonic substrates 223 are arranged in the D2 direction is exemplified, the ultrasonic substrates 223 may be configured to be laid in an array shape in the D1 direction and the D2 direction. Even in such a case, the ultrasonic substrates 223 are individually independent and are interlocked together in the elastic second sheet main body portion 221. Therefore, the flexibility of the second sheet portions 22 can be retained.

FIG. 3 is a cross-sectional view taken along line A-A in FIG. 2. FIG. 3 is a schematic cross-sectional view of a position where the ultrasonic substrate 223 of the second sheet portion 22 is provided.

As illustrated in FIG. 3, the ultrasonic substrate 223 includes an element substrate 41 which is disposed on the drive surface 221B side of the second sheet portion 22, a sealing plate 42 which is disposed on the rear surface (the connection surface 221A side) of the element substrate 41, and a wiring substrate 225 to which the element substrate 41 and the sealing plate 42 are fixed.

### Configuration of Element Substrate 41

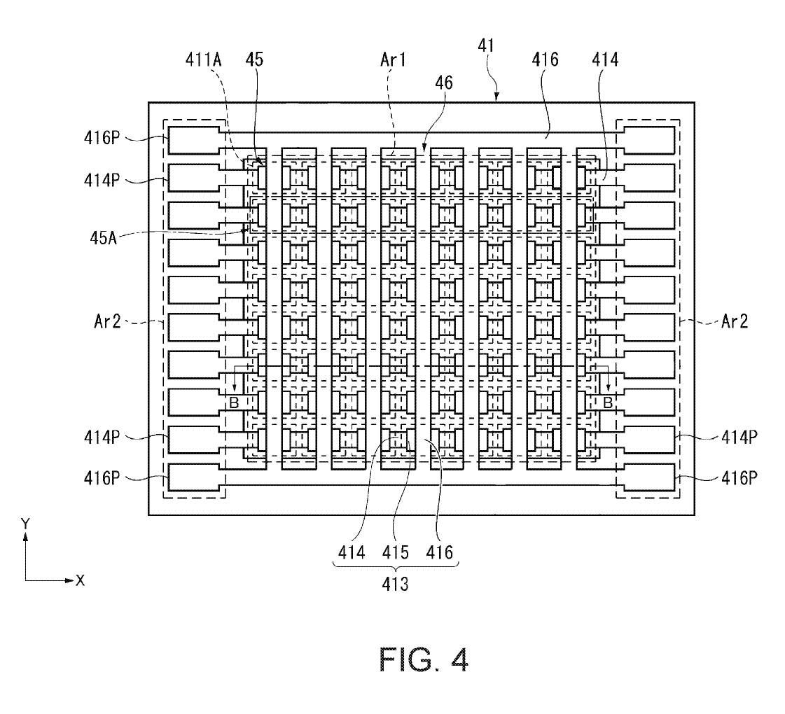

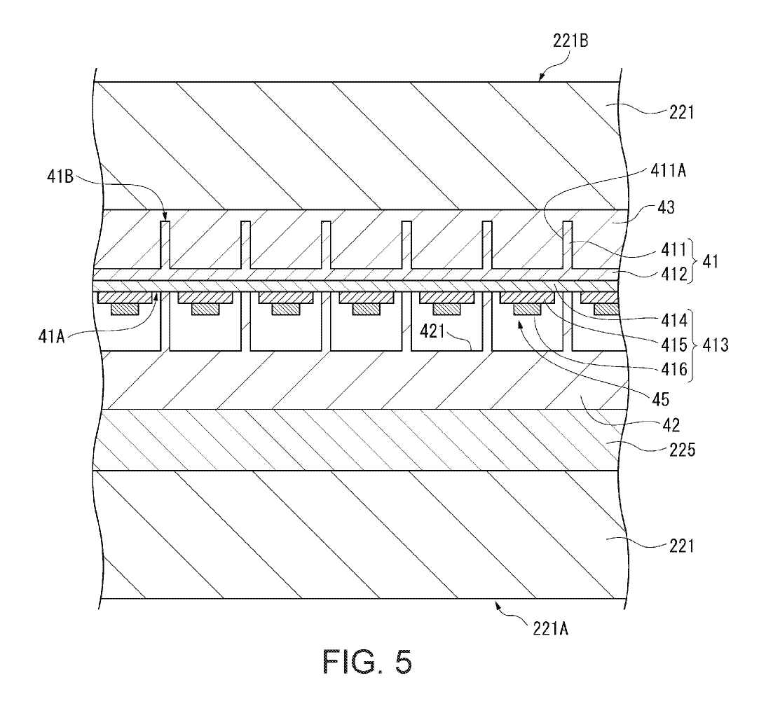

FIG. 4 is a plan view illustrating a schematic configuration of the element substrate 41. FIG. 5 is a sectional view of the element substrate 41, the sealing plate 42, and the wiring substrate 225 taken along line B-B in FIG. 4.

As illustrated in FIG. 5, the element substrate 41 includes a substrate main body portion 411, a vibration membrane 412 which is provided on the sealing plate 42 side of the substrate main body portion 411, and piezoelectric elements 413 which are laminated on the vibration membrane 412. Here, in the subsequent description, a surface facing the sealing plate 42 of the element substrate 41 will be referred to as a rear surface 41A, and a surface on a side opposite to the rear surface 41A will be referred to as an operation surface 41B. In a plan view of the element substrate 41 when viewed in a direction of the substrate thickness, the central area of the element substrate 41 becomes an array area Ar1, and a plurality of ultrasonic transducers 45 are disposed in the array area Ar1 in matrix.

For example, the substrate main body portion 411 is a semiconductor substrate made from Si and the like. Opening portions 411A respectively corresponding to the ultrasonic transducers 45 are provided within the array area Ar1 of the substrate main body portion 411. Each of the opening portions 411A is blocked by the vibration membrane 412 which is provided on the rear surface 41A side of the substrate main body portion 411.

For example, the vibration membrane 412 is configured to be made from SiO₂ or a lamination body of SiO₂ and ZrO₂ and is provided while covering the entirety of the rear surface 41A side of the substrate main body portion 411. The thickness dimension of the vibration membrane 412 is a thickness dimension which is sufficiently smaller than that of the substrate main body portion 411. In a case where the substrate main body portion 411 is configured to be made from Si and the vibration membrane 412 is configured to be made from SiO₂, the vibration membrane 412 having the desired thickness dimension can be easily formed by performing oxidation treatment of the rear surface 41A side of the substrate main body portion 411, for example. In this case, the opening portions 411A can be easily formed by performing etching processing of the substrate main body portion 411 while the vibration membrane 412 of SiO₂ serves as an etching stopper.

As illustrated in FIG. 5, the piezoelectric elements 413 each of which is a lamination body of a lower electrode 414, a piezoelectric film 415, and an upper electrode 416 are provided on the vibration membrane 412 which blocks each of the opening portions 411A. Here, one ultrasonic transducer 45 is configured to include the vibration membrane 412 blocking the opening portion 411A, and the piezoelectric element 413.

In such an ultrasonic transducer 45, a rectangular-wave voltage having a predetermined frequency is applied between the lower electrode 414 and the upper electrode 416. Therefore, the vibration membrane 412 within the opening area of the opening portion 411A is caused to vibrate, and thus, an ultrasonic wave can be sent out.

In the present embodiment, as illustrated in FIG. 4, a plurality of the above-described ultrasonic transducers 45 are disposed at predetermined places within the array area Ar1 of the element substrate 41 along an X-direction (slice direction) and a Y-direction (scanning direction) intersecting (in the present embodiment, orthogonal) the X-direction, thereby being configured to form an ultrasonic array 46.

Here, the lower electrode 414 is formed linearly along the X-direction. In other words, the lower electrode 414 is provided so as to straddle the plurality of ultrasonic transducers 45 which are in line along the X-direction. In terminal areas Ar2 outside the array area Ar1, first electrode pads 414P are configured to be formed. For example, the first electrode pads 414P are connected to a terminal portion provided in the wiring substrate 225 through a penetration electrode 422 (refer to FIG. 3) provided in the sealing plate 42.

Meanwhile, the upper electrodes 416 are connected to each other through the piezoelectric elements 413 within the array area Ar1, and portions thereof are drawn out to the terminal areas Ar2, thereby being configured to form second electrode pads 416P. For example, the second electrode pads 416P are connected to the terminal portion of the wiring substrate 225 through the penetration electrode 422 provided in the sealing plate 42.

The present embodiment illustrates an example in which the electrode pads 414P and 416P are connected to the wiring substrate 225 through the penetration electrode 422. However, for example, the electrode pads 414P and 416P may be connected thereto through FPC or the like.

In such a configuration, the ultrasonic transducers which are interlocked to each other in line in the X-direction by the lower electrode 414 are connected to each other in parallel. Here, in the present embodiment, the ultrasonic transducers 45 are configured to be one ultrasonic transducer group 45A, and a plurality of the ultrasonic transducer groups 45A in line along the Y-direction are configured to be the ultrasonic array 46 having a one-dimensional array structure.

In the present embodiment, the X-direction coincides with the longitudinal direction (D2 direction) of the second sheet portions 22. Accordingly, in the present embodiment, the output angle of an ultrasonic wave can be changed along the Y-direction (the scanning direction) intersecting the X-direction by delaying the drive pulse (drive voltage signal) input to each ultrasonic transducer group 45A. In other words, an ultrasonic wave can be output from each of the ultrasonic substrates 223 in a substantially fan-shaped output range while having the ultrasonic substrate 223 as the center, within a plane including the D1 direction orthogonal to the longitudinal direction of the second sheet portions 22 and a normal line direction of the ultrasonic substrates 223. In such a configuration, an ultrasonic wave can also be output to a space between the second sheet portions 22 and 22 adjacent to each other.

An acoustic matching layer 43 is provided on the operation surface 41B side of the substrate main body portion 411 in the element substrate 41. The acoustic matching layer 43 is configured to be made from a material of which the acoustic impedance is between the acoustic impedance of the element substrate 41 and the acoustic impedance of a living body. For example, the acoustic matching layer 43 can be configured to be made from silicone. More specifically, in the present embodiment, ultrasonic waves output from the ultrasonic transducers 45 of the element substrate 41 are output to the inside of a living body via the acoustic matching layer 43 and the second sheet main body portions 221 that are configured to be made from synthetic rubber and the like. In this case, as the second sheet main body portion 221, it is preferable to use synthetic rubber of which the value of the acoustic impedance is close to that of the acoustic impedance of a living body while being slightly greater than that of the acoustic impedance of a living body. It is preferable that the value of the acoustic impedance of the acoustic matching layer 43 is between those of the acoustic impedance of the element substrate 41 (ultrasonic transducers 45) and the acoustic impedance of the second sheet main body portions 221.

Such an acoustic matching layer 43 fills the insides of the opening portions 411A of the element substrate 41 and is formed to have a predetermined thickness dimension from the operation surface 41B side of the substrate main body portion 411. In such a configuration, no air layer is interposed inside the opening portions 411A, and reflection or attenuation of an ultrasonic wave caused by the air layer can be suppressed. A surface on a side opposite to the element substrate 41 of the acoustic matching layer 43 becomes a uniformly flat surface and is in close contact with the synthetic rubber-made second sheet main body portions 221. Accordingly, no air layer is interposed between the acoustic matching layer 43 and the second sheet main body portions 221, and reflection or attenuation of an ultrasonic wave caused by the air layer can be suppressed.

The present embodiment illustrates an example in which the acoustic matching layer 43 and the second sheet main body portions 221 are in close contact with each other. However, an acoustic lens may be provided between the acoustic matching layer 43 and the second sheet main body portions 221. In this case, an ultrasonic wave having a desired beam shape can be output due to the acoustic lens.

### Configuration of Sealing Plate 42

For example, the sealing plate 42 is formed to have the same flat surface shape when viewed in the thickness direction as the element substrate 41 and is configured to be a semiconductor substrate such as Si, or an insulating substrate. The material and the thickness of the sealing plate 42 affect the characteristics of frequency of the ultrasonic transducers 45. Therefore, it is preferable that the material and the thickness are set based on the center frequency of an ultrasonic wave transceived by the ultrasonic transducers 45.

In the sealing plate 42, the plurality of recessed grooves 421 corresponding to the opening portions 411A of the element substrate 41 are formed in the array-facing area facing the array area Ar1 of the element substrate 41. Accordingly, gaps having predetermined dimensions are provided between the vibration membrane 412 and the element substrate 41 at areas caused to vibrate by the ultrasonic transducers 45 (inside the opening portions 411A) in the vibration membrane 412. Thus, vibration of the vibration membrane 412 is not hindered. In addition, it is possible to prevent inconvenience (crosstalk) in that a back wave from one ultrasonic transducer 45 is incident on an adjacent ultrasonic transducer 45.

When the vibration membrane 412 vibrates, an ultrasonic wave is also released to the sealing plate 42 side (the rear surface 41A side) as a back wave in addition to the opening portions 411A side (the operation surface 41B side). The back wave is reflected by the sealing plate 42 and is released to the vibration membrane 412 side again via the gaps. In this case, when the phases of the reflected back wave and an ultrasonic wave released from the vibration membrane 412 to the operation surface 41B side are misaligned, the ultrasonic wave is attenuated. Therefore, in the present embodiment, the groove depth of each recessed groove 421 is set such that the acoustic distance in the gap becomes odd-number times the quarter of a wavelength λ(λ/4) of an ultrasonic wave. In other words, in consideration of the wavelength λ of the ultrasonic wave emitted from the ultrasonic transducers 45, the thickness dimensions of each portion of the element substrate 41 and the sealing plate 42 are set.

At positions facing the terminal areas Ar2 of the element substrate 41, the sealing plate 42 has opening portions (not illustrated) so as to respectively correspond to the electrode pads 414P and 416P provided in the terminal areas Ar2. The penetration electrodes 422 (through-silicon via: TSV) penetrating the sealing plate 42 in the thickness direction are provided in the opening portions. As described above, each of the electrode pads 414P and 416P is connected to the terminal portion in the wiring substrate 225 via the penetration electrodes 422.

### Configuration of Wiring Substrate 225

The wiring substrate 225 has a terminal portion (not illustrated) which is electrically connected to each of the electrode pads 414P and 416P through the penetration electrodes 422. A drive control circuit 226 (refer to FIG. 3) and the like for driving each of the ultrasonic transducers 45 are provided in the wiring substrate 225. The drive control circuit 226 includes a pulser which outputs the drive pulse applied to each of the ultrasonic transducers 45, at a predetermined cycle. The drive control circuit 226 includes a microcomputer which controls an operation of the pulser, selects the ultrasonic transducer 45 which becomes the output destination of the drive pulse, and controls the output direction of an ultrasonic wave by delaying driving of the ultrasonic transducer 45.

Moreover, the wiring substrate 225 is provided with a terminal portion (not illustrated) connected to the drive control circuit 226 and the signal lines 224 branched from the branch circuit portions 222 are connected to the terminal portion. Accordingly, the drive control circuit 226 of the wiring substrate 225 can acquire the drive command data input from the control device 3, and can output ultrasonic waves from the ultrasonic substrates 223 by driving each ultrasonic transducer 45 based on the drive command data.

### Configuration of Control Device 3

As illustrated in FIG. 1, the control device 3 is configured to include an operation unit 31, a display unit 32, an I/F unit 33 (interface unit), a storage unit 34, and a computation unit 35.

The operation unit 31 outputs an operation signal to the computation unit 35 in response to an input operation of a user.

For example, the display unit 32 displays an operation guide of the ultrasonic apparatus 1 for a user based on the control of the computation unit 35.

The I/F unit 33 is connected to the ultrasonic probe 2 via the cable 10 so as to be able to communicate with the ultrasonic probe 2.

The storage unit 34 stores various types of programs and various types of data for controlling the ultrasonic apparatus 1.

As the data stored in the storage unit 34, for example, a plurality of items of sequential data indicating a drive method of the ultrasonic probe 2 with respect to the treatment site or contents of treatment of a living body are stored, for example, in a table data structure.

For example, the sequential data includes target data, drive element data, and output range data.

The target data is data related to a target subject which is an output target of an ultrasonic wave, that is, data indicating the treatment site or the contents of treatment of a living body. For example, a treatment ID corresponding to the treatment site or the contents of treatment is recorded. For example, in cases of the treatment ID in which the abdomen is the treatment site and combustion assistance of visceral fat in the abdomen is the contents of treatment, the treatment ID in which the abdomen is the treatment site and improvement of muscular pain in the abdominal muscle is the contents of treatment, and the treatment ID in which the abdomen is the treatment site and skin care for a skin surface is the contents of treatment, the treatment IDs different from each other are respectively applied.

The drive element data is data indicating the position of the to-be-driven ultrasonic transducer 45 with respect to the treatment ID. In the present embodiment, for example, a sheet ID is applied to each second sheet portion 22, an array ID is applied to each ultrasonic substrate 223, and an element ID is applied to each ultrasonic transducer 45. In the drive element data, the sheet ID and the array ID of the second sheet portion 22 and the ultrasonic substrate 223 including the to-be-driven ultrasonic transducer 45, and the element ID of the ultrasonic transducer 45 in the ultrasonic substrates 223 are recorded so as to correspond to each treatment ID.

The output range data is data indicating a range of outputting an ultrasonic wave, with respect to the treatment ID. In the output range data, the output time of the drive pulse with respect to the ultrasonic transducer 45 designated in the drive element data is recorded.

In other words, in the present embodiment, as described above, an ultrasonic wave is output in a desired direction by controlling the delay time of the output time of the drive pulse with respect to the plurality of ultrasonic transducer groups 45A in line along the scanning direction. Therefore, the output time (delay time) of the drive pulse output with respect to each ultrasonic transducer 45 is recorded in the output range data, and the ultrasonic transducer 45 is driven based on the output time. Accordingly, it is possible to control the output range of an ultrasonic wave.

As the output range data, an angle of a fan-shaped output range of an ultrasonic wave having the ultrasonic substrate 223 as the center may be recorded. In this case, the delay time is calculated by the drive control circuit 226 (microcomputer) provided in each ultrasonic substrate 223, or the computation unit 35 of the control device 3, and then, each ultrasonic transducer 45 may be sequentially driven at the calculated delay time.

As the sequential data, other types of data may be included.

For example, a drive order and the like of the second sheet portion 22 and the ultrasonic substrate 223 (ultrasonic array 46) of the drive target may be recorded. For example, data such as a signal level and a pulse width (duty ratio) of the drive pulse output to the ultrasonic transducer 45 may be recorded.

For example, the computation unit 35 is configured to include a central processing unit (CPU), a storage circuit, and the like. The computation unit 35 executes various types of processing by reading various types of programs stored in the storage unit 34, for example. Specifically, as illustrated in FIG. 1, the computation unit 35 reads and executes various types of programs, thereby functioning as a sequence acquisition unit 351, a drive control unit 352, and the like.

For example, the sequence acquisition unit 351 acquires the sequential data corresponding to the treatment site or the contents of treatment from the sequential data stored in the storage unit 34, based on an operation signal input from the operation unit 31.

The drive control unit 352 controls the ultrasonic probe 2 based on the acquired sequential data. As illustrated in FIG. 1, the drive control unit 352 functions as an element selection unit 352A, a drive parameter setting unit 352B, and a drive command unit 352C.

The element selection unit 352A selects the to-be-driven ultrasonic transducer 45 based on the drive element data of the sequential data.

The drive parameter setting unit 352B generates the drive command data in which the drive time of each ultrasonic transducer 45 is set, based on the output range data.

The drive command unit 352C outputs the generated drive command from the I/F unit 33 to the ultrasonic probe 2.

### Drive Method of Ultrasonic Apparatus 1

### Adherently Fixing Ultrasonic Probe 2

Subsequently, a drive method of the above-described ultrasonic apparatus 1 will be described with reference to the drawings.

In a case where the ultrasonic apparatus 1 is used, a user adherently fixes the ultrasonic probe 2 to a desired position in a living body.

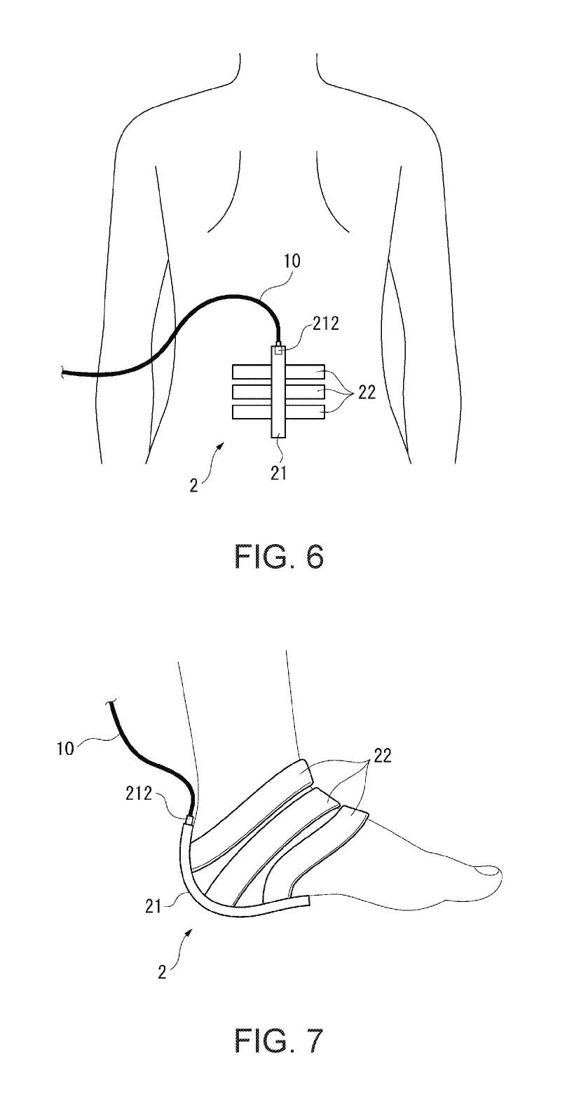

Each of FIGS. 6 and 7 is an example illustrating a method of fixing the ultrasonic probe 2 to a living body.

As illustrated in FIG. 6, in a case where the ultrasonic probe 2 is fixed to the back, a user adherently fixes the central portion of the drive surface 221B of the second sheet main body portions 221 in the second sheet portions 22 to a position along the backbone of the back in a state where the first sheet portion 21 follows the backbone.

Each second sheet portion 22 is caused to follow the back from the central portion toward the tip portion, thereby adhering to the back. Accordingly, the drive surface 221B of the second sheet main body portions 221 can be fixed to the back in a close contact manner.

Here, in a case where a rectangular ultrasonic probe having a large size is adherently applied to the back so as to cover the back, it is difficult to fix the ultrasonic probe such that air bubbles do not enter a space between the ultrasonic probe and a living body. Particularly, in a case where the ultrasonic probe adheres to a place such as the back which is difficult for a user oneself to visually recognize, it is extremely difficult to cause the ultrasonic probe to adhere to a predetermined position such that air bubbles are not interposed therebetween, without using other's help. In contrast, in the present embodiment, as described above, a plurality of the longitudinal second sheet portions 22 are caused to adhere to a living body in order to be oriented from the central position toward the tip portion. In this case, one direction along the longitudinal direction of the second sheet portions 22 maybe caused to follow along the skin surface of a living body. Thus, compared to a case where a large-sized ultrasonic probe is caused to adhere, it is possible to easily and reliably cause the drive surface 221B to adhere to a living body.

The second sheet main body portions 221 are configured to be made from elastic synthetic rubber. Therefore, the second sheet main body portions 221 can be stretched in accordance with the individual size of a user.

The example illustrated in FIG. 7 is an example in which the ultrasonic probe 2 is fixed to the heel of a foot.

In this example, after the first sheet portion 21 is brought into contact with the heel and is positioned thereat, the second sheet portions 22 is fixed to the back of the foot so as to be wound around the foot.

FIG. 7 illustrates an example in which the ultrasonic probe 2 is fixed to the heel of a foot. However, in addition thereto, the second sheet portions 22 can be adherently fixed to a curved portion of a living body such as a leg, an arm, a shoulder, and the waist (side portion) so as to wrap around a treatment target site.

### Drive Method of Ultrasonic Apparatus 1

Subsequently, the drive method of the ultrasonic apparatus 1 will be described.

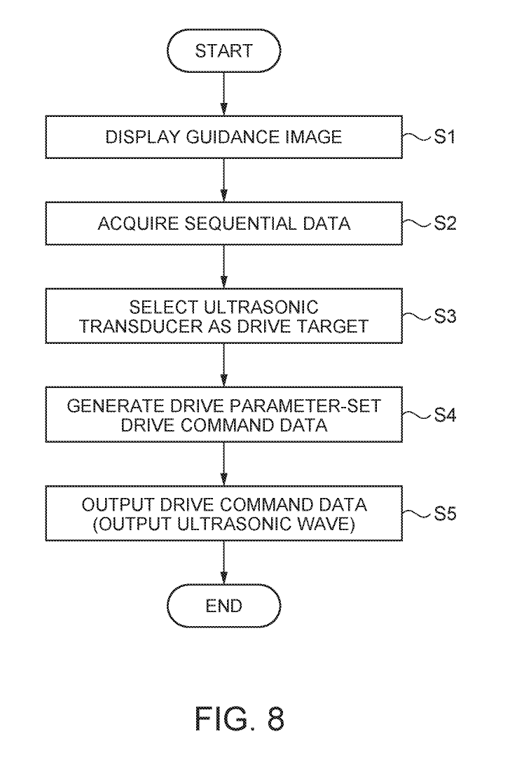

FIG. 8 is a flow chart illustrating a treatment method of a treatment site performed by using the ultrasonic apparatus 1.

First, the computation unit 35 of the ultrasonic apparatus 1 causes the display unit 32 to display a guidance image such that a user inputs the treatment site or the contents of treatment (Step S1). As the guidance image, for example, it is preferable to display guidance for a user to select the treatment site or the contents of treatment based on the sequential data.

When the user operates the operation unit 31, and an input operation of inputting the treatment site or the contents of treatment is performed, an operation signal based on the input operation is input from the operation unit 31 to the computation unit 35. For example, the operation signal includes the treatment site or the contents of treatment which is selected.

When an operation signal is input, the sequence acquisition unit 351 of the computation unit 35 searches for and acquires the sequential data stored in the storage unit 34, by using the treatment site and the contents of treatment included in the operation signal (Step S2).

In this case, notification may be issued by causing the display unit 32 to display information related to adherently fixing the ultrasonic probe 2. In this case, guidance data (image or voice) indicating an appropriate adhesional position and an appropriate adhesion method with respect to the treatment site is recorded by associating with the sequential data stored in the storage unit 34. When the sequential data is acquired through Step S2, notification of the guidance data which has been associated with the sequential data is issued. In this case, when ultrasonic treatment is executed based on the subsequent sequential data, it is highly possible for the ultrasonic probe 2 to be adherently applied to a position at which an optimum treatment effect can be obtained. Thus, it is possible to further obtain the treatment effect.

After Step S2, the element selection unit 352A of the drive control unit 352 selects the ultrasonic transducer 45 which becomes the drive target, based on the acquired sequential data (Step S3). Accordingly, the ultrasonic transducer 45 which is at an optimum position and has the optimum number of openings (the number of the drive elements of the ultrasonic transducer 45) corresponding to the treatment site or the contents of treatment input by a user is selected.

The drive parameter setting unit 352B of the drive control unit 352 generates the drive command data in which a drive time (delay time) with respect to the ultrasonic transducer 45 selected through Step S2 is set, based on the output range data (Step S4). In a case where the second sheet portions 22 of the drive target, the drive order of the ultrasonic array 46, the signal level (voltage value) of the drive pulse, the pulse width, and the like are included as the sequential data, these parameters may be included in the drive command data.

Thereafter, the drive command unit 352C of the drive control unit 352 outputs the drive command data generated from the I/F unit 33 through Step S4, to the ultrasonic probe 2 (Step S5).

Accordingly, the drive command data is output to the drive control circuit 226 provided from the first sheet portion 21 to each wiring substrate 225 of each second sheet portion 22. The drive control circuit 226 individually drives the ultrasonic transducers 45 (ultrasonic transducer group 45A) which become the drive target based on the drive command data at the drive time set as the drive parameters. Accordingly, an ultrasonic wave is output within the set output range from each ultrasonic substrate 223 of the second sheet portions 22.

Operational effect of Present Embodiment

The ultrasonic apparatus 1 according to the present embodiment has the ultrasonic probe 2 and the control device 3. The ultrasonic probe 2 includes the longitudinal first sheet portion 21 and the second sheet portions 22 provided in a direction of being separated from the first sheet portion 21. Each second sheet portion 22 has the connection surface 221A connected to the first sheet portion 21, and the drive surface 221B on a side opposite to the connection surface 221A. The plurality of ultrasonic transducers 45 outputting ultrasonic waves are provided on the drive surface 221B side.

In such a configuration, the plurality of second sheet portions 22 are configured to be provided with respect to the first sheet portion 21. Therefore, each second sheet portion 22 is caused to adhere to the treatment site. Accordingly, without allowing air to be interposed between the second sheet portions 22 and a living body, the ultrasonic probe 2 can be fixed to an appropriate position in the living body.

In other words, in a case where a large-sized sheet-like ultrasonic probe is caused to adhere to a living body across the treatment site, the curved shape of the surface of the living body varies depending on the treatment site, the individual difference, and the like. Accordingly, it has been difficult to cause the ultrasonic probe to be adherently fixed to the treatment position such that air bubbles are not interposed between the living body and the ultrasonic probe. In contrast, in the present embodiment, in a state where the first sheet portion 21 is caused to follow along the central position at the treatment site, the second sheet portions 22 is caused to adhere thereto in order in the direction of being separated from the first sheet portion 21. In this case, the second sheet portions 22 may be caused to follow along the curved shape of the living body in the linear direction along the longitudinal direction. Thus, air bubbles (air layer) are unlikely to enter a space between the living body and the second sheet portions 22. Even though air bubbles are generated, the air bubbles are likely to be pressed out. Accordingly, an ultrasonic wave can be appropriately propagated to the living body. When the plurality of second sheet portions 22 are provided, it is easy to cope with an undulating living body, and thus, the characteristics of close contact increase.

Moreover, depending on the treatment site of the living body, there are cases where the output value of an ultrasonic wave needs to be weakened in order to avoid destruction of biological tissue. In the present embodiment, for example, some of the plurality of second sheet portions 22 are not adherently fixed to a living body and only the remaining second sheet portions 22 can be adherently fixed to the living body. In this case, an ultrasonic wave can be output from only the adherently fixed second sheet portions 22 to the living body. Thus, it is possible to suitably adjust the intensity of the ultrasonic wave.

In the present embodiment, the second sheet portions 22 are line-symmetrical while having the first sheet portion 21 as the center.

Generally, a living body has many portions which are configured to be bilateral symmetry with respect to the central axis such as the backbone. In the present embodiment, the first sheet portion 21 is positioned by being caused to follow the central axis (the backbone or the like) of a living body. Then, the second sheet portions 22 are bilaterally spread and are adherently fixed thereto. Thus, the ultrasonic probe 2 can be easily caused to adhere an appropriate treatment site.

In the present embodiment, in the second sheet portions 22, the signal lines 224 with respect to each ultrasonic substrate 223 provided with the plurality of ultrasonic transducers 45 are disposed while meandering in the longitudinal direction of the second sheet portions.

Accordingly, when the second sheet portions 22 are caused to adhere, even in a case where stress is applied such that the second sheet main body portions 221 extend in the longitudinal direction, the signal lines 224 are unlikely to be disconnected. Thus, an improvement of reliability can be achieved.

In the present embodiment, in each ultrasonic substrate 223, a plurality of the ultrasonic transducer groups 45A to each of which the plurality of ultrasonic transducers 45 are connected together in parallel are arranged in the scanning direction.

In such a configuration, an ultrasonic wave having significant acoustic pressure can be output from each ultrasonic transducer 45 of the ultrasonic transducer group 45A.

In a case where an ultrasonic wave is received by the ultrasonic transducers 45, it is desirable that the ultrasonic transducers 45 are configured to be connected in series. In a case where both outputting and receiving of an ultrasonic wave are performed in the ultrasonic probe 2, there is a need to have a switching circuit for switching between a circuit configuration in which the ultrasonic transducers are connected together in parallel and a circuit configuration in which the ultrasonic transducers 45 are connected together in series. In contrast, in the ultrasonic apparatus 1 of the present embodiment, receiving processing is not performed in each ultrasonic transducer 45. Therefore, as described above, the circuit configuration in which the ultrasonic transducers 45 are connected together in parallel may be provided. Accordingly, it is possible to achieve the simplified circuit configuration.

In the ultrasonic apparatus 1 according to the present embodiment, the storage unit 34 of the control device 3 stores items of the sequential data corresponding to a plurality of items of the treatment site or the contents of treatment. When the sequence acquisition unit 351 acquires the sequential data corresponding to the treatment site, the drive control unit 352 outputs the drive command data based on the acquired sequential data to the ultrasonic probe 2. Accordingly, the ultrasonic probe 2 outputs an ultrasonic wave from the ultrasonic probe 2 through the output procedure corresponding to the treatment site.

Accordingly, an ultrasonic wave having optimum intensity corresponding to the treatment site can be output. Thus, it is possible to perform safe ultrasonic treatment while avoiding the risk of destruction or the like of biological tissue.

The drive command data includes the drive time based on the output range data, and the plurality of ultrasonic transducer groups 45A are sequentially delayed and driven based on the drive time. Accordingly, it is possible to efficiently output an ultrasonic wave within an optimum range corresponding to the treatment site. For example, an ultrasonic wave can be output in a narrow range with respect to a portion having a narrow range such as an arm, and an ultrasonic wave can be output in a wide range with respect to a wide range such as the back.

### Second Embodiment

Subsequently, a second embodiment of the invention will be described with reference to the drawings.

The first embodiment has exemplified a configuration in which an ultrasonic wave having the intensity corresponding to the treatment site or the contents of treatment is output by suitably selecting the to-be-driven ultrasonic transducer 45 (ultrasonic transducer group 45A) in accordance with the treatment site. In contrast, the second embodiment includes the plurality of ultrasonic transducers 45 of which the output ultrasonic waves are different from each other in intensity. The second embodiment is different from the first embodiment in that selection is made among the plurality of ultrasonic transducers 45.

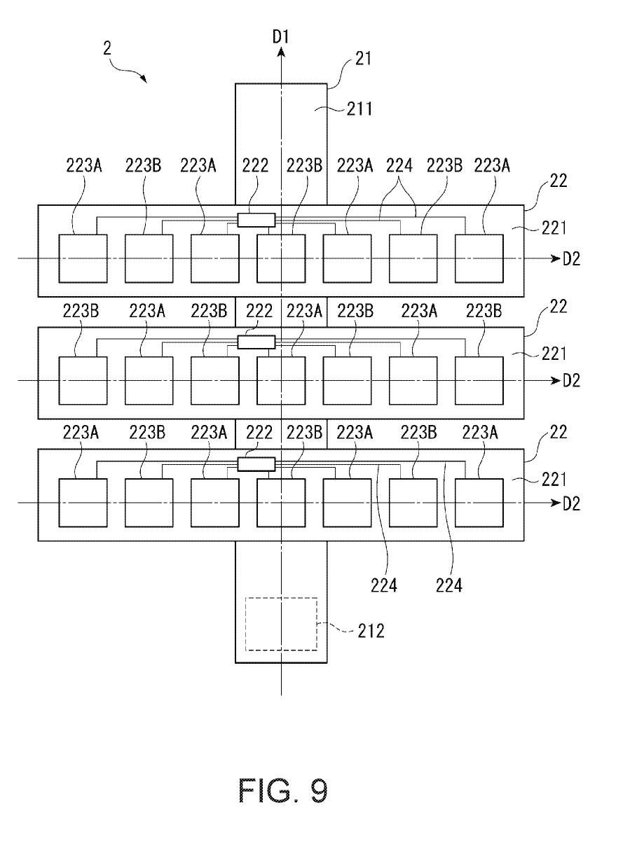

FIG. 9 is a plan view of an ultrasonic probe 2 according to the second embodiment.

In FIG. 9, in the second sheet portions 22 of the ultrasonic probe 2, first ultrasonic substrates 223A and second ultrasonic substrates 223B are alternately disposed along the longitudinal direction (D2 direction).

The first ultrasonic substrates 223A and the second ultrasonic substrates 223B have configurations similar to those of the ultrasonic substrates 223 in the first embodiment. However, the ultrasonic transducers 45 are different from each other in size. In other words, openings of the opening portions 411A provided in the element substrate 41 are different from each other in size.

For example, the opening width dimension of each opening portion 411A is M and an ultrasonic wave having a first frequency is output in the first ultrasonic substrates 223A. In contrast, in the second ultrasonic substrates 223B, the opening dimension of each opening portions 411A is N (N<M) and an ultrasonic wave having a second frequency greater than the first frequency is output.

Generally, the intensity I (W/cm₂) of an ultrasonic wave becomes stronger as the frequency of an output ultrasonic wave becomes higher. As the frequency of an ultrasonic wave becomes lower, the ultrasonic wave is unlikely to be attenuated, and thus, the ultrasonic wave can be propagated to a deep portion in a living body. Therefore, in the above-described example, ultrasonic waves having the second frequencies output from the second ultrasonic substrates 223B having the small opening dimensions of the opening portions 411A are output to places in the vicinity of the surface layer of a living body at strong intensity. Meanwhile, even though ultrasonic waves having the first frequencies output from the first ultrasonic substrates 223A are weak in intensity of the ultrasonic wave, the ultrasonic waves can be propagated to a deep portion in a living body.

In the present embodiment, when ultrasonic treatment is performed by using the ultrasonic probe 2, the computation unit 35 of the control device 3 functions as the sequence acquisition unit 351 and the drive control unit 352, similar to the first embodiment. Therefore, the element selection unit 352A of the drive control unit 352 selects the ultrasonic transducer 45 (ultrasonic transducer group 45A) as the drive target based on the drive element data included in the sequential data.

For example, in a case where the contents of treatment are combustion assistance of visceral fat, the array ID and the element ID of each first ultrasonic substrate 223A are recorded as the drive element data. In a case where the contents of treatment are skin care, hair growth promotion, and the like, the array ID and the element ID of each second ultrasonic substrate 223B are recorded as the drive element data.

Accordingly, through the drive method similar to that of the first embodiment, it is possible to output an ultrasonic wave having optimum output and frequency with respect to the treatment site and the contents of treatment.

### Operational Effect of Present Embodiment

The ultrasonic probe 2 according to the present embodiment includes the first ultrasonic substrates 223A provided with the ultrasonic transducers 45 (first ultrasonic transducers) which can output ultrasonic waves having the first frequencies, and the second ultrasonic substrates 223B provided with the ultrasonic transducers 45 (second ultrasonic transducers) which can output ultrasonic waves having the second frequencies greater than the first frequencies.

In such a configuration, in accordance with the treatment site or the contents of treatment of transmitting an ultrasonic wave performed by the ultrasonic probe 2, it is possible to select (switch between) the ultrasonic substrates 223A and 223B outputting ultrasonic waves. Accordingly, an optimum ultrasonic wave corresponding to the treatment site or the contents of treatment can be output. Thus, for example, it is possible to avoid the risk of destruction or the like of biological tissue caused by an ultrasonic wave.

In the description above, an example in which the first ultrasonic substrates 223A and the second ultrasonic substrates 223B output ultrasonic waves different from each other is illustrated. However, the embodiment is not limited thereto. For example, one ultrasonic substrate 223 may be configured to be provided with the first ultrasonic transducers outputting ultrasonic waves having the first frequencies and the second ultrasonic transducers outputting ultrasonic waves having the second frequencies. In this case, ultrasonic waves having each of the frequencies can be uniformly output due to the configuration in which the ultrasonic transducer groups 45A of the first ultrasonic transducers and the ultrasonic transducer groups 45A of the second ultrasonic transducers are alternately disposed along the scanning direction of the element substrate 41.

### Third Embodiment

Subsequently, a third embodiment of the invention will be described with reference to the drawings.

In the ultrasonic apparatus 1 according to the first embodiment, a configuration in which the second sheet portions 22 are glued and fixed to the first sheet portion 21 and power is supplied to the ultrasonic substrates 223 due to the connection wiring portions 213 wired from the first sheet portion 21 to the branch circuit portions 222 of the second sheet portions 22 has been exemplified. In contrast, the third embodiment is different from the first embodiment in the point that the second sheet portions 22 are attachable/detachable with respect to the first sheet portion 21 and power is supplied in a contactless manner.

In the subsequent description, the contents which have already been described above will not be repeatedly described or will be simplified.

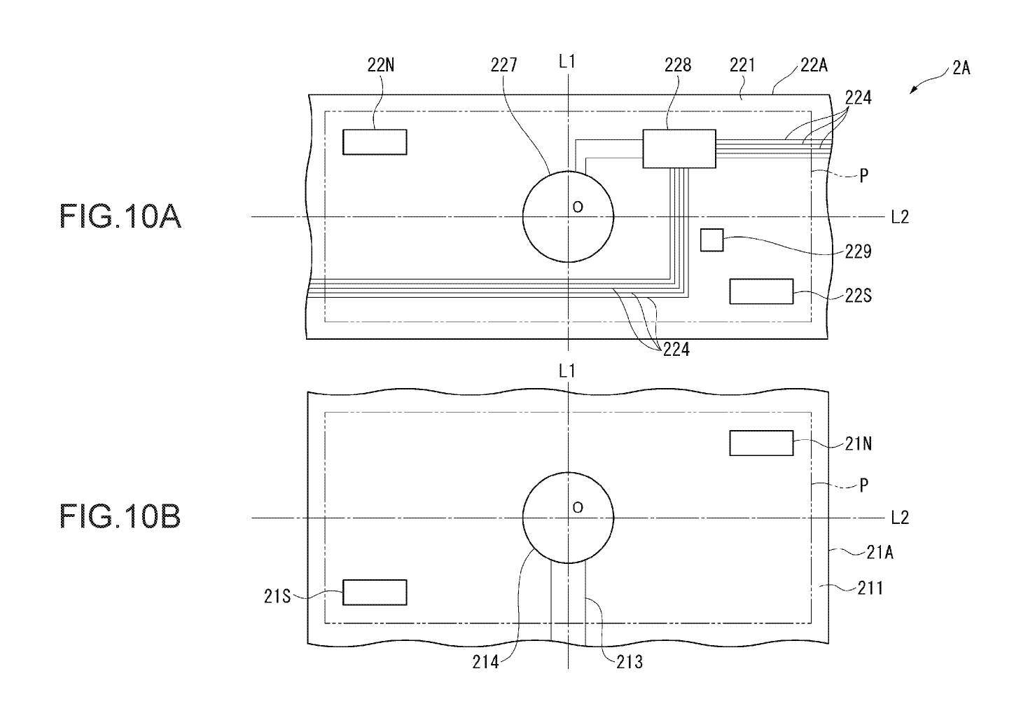

FIG. 10A is a plan view of a second sheet portion 22A in the vicinity of the connection portion with respect to a first sheet portion 21A when viewed from the connection surface 221A side. FIG. 10B is a plan view of the first sheet portion 21A in the vicinity of the connection portion with respect to the second sheet portion 22A when viewed from the surface side where the second sheet portion 22A is attached. The two-dot chain line indicates an attachment area P (position of connection) where the first sheet portion 21A and the second sheet portion 22A overlap each other.

In an ultrasonic probe 2A according to the present embodiment, the first sheet portion 21A and the second sheet portion 22A are independently provided, and power is supplied to each ultrasonic substrate 223 of the second sheet portion 22A through a contactless power transmission mechanism provided in the attachment area P. The contactless power transmission mechanism is configured to include a power transmitting coil 214 provided in the first sheet portion 21A, and a power receiving coil 227 provided in the second sheet portion 22A.

The power transmitting coil 214 is provided at a central position in the attachment area P inside the first sheet main body portion 211 of the first sheet portion 21A. More specifically, the power transmitting coil 214 is configured to have a predetermined diameter dimension R while having an intersection position O between a central axial line L1 along the longitudinal direction (D1 direction) of the first sheet portion 21A and a central axial line L2 along the longitudinal direction (D2 direction) of the second sheet portion 22A attached to the attachment area P, as the center. The power transmitting coil 214 is connected to the connector portion 212. Similar to the first embodiment, the connector portion 212 may be connected to the control device 3 or may be connected to a different power supply unit (for example, a power supply outlet for home use).

Meanwhile, the power receiving coil 227 is provided at the central position in the attachment area P inside the second sheet main body portion 221 of the second sheet portion 22A. More specifically, the power receiving coil 227 is configured to have the same diameter dimension R as the power transmitting coil 214 while having the intersection position O between the central axial line L2 along the longitudinal direction (D2 direction) of the second sheet portion 22A and the central axial line L1 along the longitudinal direction (D1 direction) of the first sheet portion 21A attached to the attachment area P, as the center. The power receiving coil 227 is connected to the control circuit unit 228 provided inside the second sheet main body portion 221.

In such a contactless power transmission mechanism, a magnetic flux is generated by causing a current to flow in the power transmitting coil 214, and electromotive force is generated in the power receiving coil 227 due to the magnetic flux.

The control circuit unit 228 includes a battery, a power supply circuit, a control circuit which controls driving of the ultrasonic substrates 223, and the like. For example, the battery is configured to include a capacitor and stores electromotive force generated in the power receiving coil. The power supply circuit supplies power stored in the battery to each ultrasonic substrate 223.

A reception unit 229 receiving the drive command data transmitted from the control device 3 is connected to the control circuit unit 228. In other words, in the present embodiment, for example, the control device 3 is provided with a wireless communication unit (not illustrated) which performs transceiving of data through infrared-ray communication, Bluetooth (registered trademark), and the like, and the reception unit 229 receives the drive command data transmitted from the wireless communication unit. The drive command data is data similar to the drive command data described in the first embodiment. The drive command data is data indicating the output procedure of an ultrasonic wave corresponding to the treatment site.

When the drive command data is input from the reception unit 229, the control circuit unit 228 outputs the drive command data to the drive control circuit 226 of each ultrasonic substrate 223. Accordingly, similar to the first embodiment, driving of the ultrasonic transducer groups 45A in each drive control circuit 226 is controlled, and an optimum ultrasonic wave corresponding to the treatment site is output from each ultrasonic substrate 223 (ultrasonic array 46).

In the present embodiment, the first sheet portion 21A is provided with first magnets 21S and 21N at the connection portion (attachment area P) between the first sheet portion 21A and the second sheet portion 22A. In the first magnet 21S, a surface on the second sheet portion 22A side becomes the S-pole. In the first magnet 21N, a surface on the second sheet portion 22A side becomes the N-pole.

More specifically, when the attachment area P is divided into four areas by the central axial line L1 and the central axial line L2, the first magnet 21S and the first magnet 21N are respectively provided within an area so as to be point-symmetrical with respect to the intersection position O between the central axial line L1 and the central axial line L2.

The second sheet portion 22A is provided with second magnets 22S and 22N at the attachment area P. In the second magnet 22S, a surface on the first sheet portion 21A side becomes the S-pole. In the second magnet 22N, a surface on the first sheet portion 21A side becomes the N-pole. The second magnet 22S is provided at a position facing the first magnet 21N in the attachment area P, and the second magnet 22N is provided at a position facing the first magnet 21S in the attachment area P, respectively.

Therefore, in a case where the second sheet portion 22A is fixed to the first sheet portion 21A, the magnets 21S and 22N, and the magnets 21N and 22S can be easily fixed together due to the magnetic force. After the ultrasonic treatment ends, the second sheet portion 22A can be easily removed by being separated from the first sheet portion 21A.

### Operational Effect of Present Embodiment

In the ultrasonic probe 2A according to the present embodiment, the second sheet portion 22A is provided so as to be attachable/detachable with respect to the first sheet portion 21A. Accordingly, for example, in a case where a portion among the plurality of second sheet portions 22A is unnecessary, the unnecessary second sheet portion 22A can be removed from the first sheet portion 21A. For example, depending on the treatment site, there are cases where the treatment site can be sufficiently covered when only a portion among the plurality of second sheet portions 22A is caused to adhere to a living body. In such a case, when the unnecessary second sheet portion 22A remains being attached, there are cases where the unnecessary second sheet portion 22A touches other positions of the living body, thereby causing the user to experience discomfort or hindering ultrasonic treatment. In contrast, in the present embodiment, since the second sheet portion 22A can be removed as described above, comfortable ultrasonic treatment can be performed and the second sheet portion 22A can be caused to appropriately adhere to the treatment site without causing a user to experience such discomfort.

When the ultrasonic probe 2 is removed from a living body, for example, an operation in which the second sheet portion 22A is gradually removed or the like can be performed. Thus, it is possible to improve the operability.

The ultrasonic probe 2A according to the present embodiment is configured to be attachable/detachable by the first magnets 21S and 21N of the first sheet portion 21A, and the second magnets 22N and 22S of the second sheet portion 22A. Accordingly, when the second sheet portion 22A is torn off in the direction of being separated from the first sheet portion 21A, the second sheet portion 22A can be easily separated from the first sheet portion 21A. The second sheet portion 22A can be easily connected and fixed to the first sheet portion 21A by only causing the second sheet portion 22A to approach the first sheet portion 21A. Particularly, the configuration is advantageous when the ultrasonic probe 2 is attached and detached with respect to a position which is out of view (for example, the back) of a user.

In the ultrasonic probe 2A according to the present embodiment, the first sheet portion 21A has the power transmitting coil 214 at the attachment area P with respect to the second sheet portion 22A, and the second sheet portion 22A has the power receiving coil 227 at the attachment area P. In such a configuration, a magnetic flux is generated by causing a current to flow in the power transmitting coil 214, and electromotive force is generated in the power receiving coil 227. Accordingly, power can be supplied to each ultrasonic substrate 223 (ultrasonic transducer 45) of the second sheet portion 22A through contactless power transmission.

### Fourth Embodiment

Subsequently, a fourth embodiment of the invention will be described with reference to the drawings.

The third embodiment illustrates an example in which the longitudinal second sheet portion 22A is attached to the first sheet portion 21A in an attachable/detachable manner. In contrast, the second sheet portion 22A may be removed, and the second sheet portion having an optimized shape with respect to the treatment site of a living body or the contents of treatment may be configured to be attached, in place thereof.

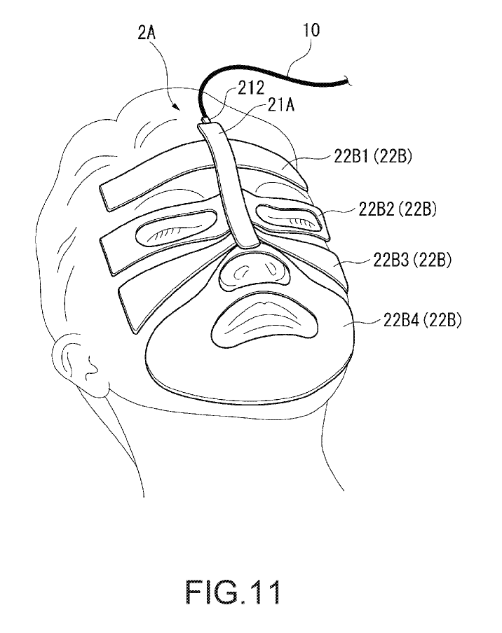

In the fourth embodiment, the ultrasonic probe 2A can function as a facial beauty instrument by attaching a second sheet portion 22B (refer to FIG. 11) which is optimized for the shape of the face, in place of the second sheet portion 22A in the third embodiment.

FIG. 11 is a plan view illustrating a schematic configuration of the ultrasonic probe 2A of the fourth embodiment.

Specifically, as illustrated in FIG. 11, the ultrasonic probe 2A includes a plurality of the second sheet portions 22B (22B1, 22B2, 22B3, and 22B4) attached to the first sheet portion 21A.

The first sheet portion 21A is configured to be longitudinal along the D1 direction, similar to that of the third embodiment. In the present embodiment, as illustrated in FIG. 11, the first sheet portion 21A is disposed along the center of the face (the ridge of the nose).

The second sheet portion 22B1 has a shape which can adhere to the forehead of the face.

The second sheet portion 22B2 has a shape in which a substantially U-shaped piece surrounding the eyes of the face is provided so as to be bilaterally symmetrical while having the first sheet portion 21A as the center.

The second sheet portion 22B3 has a shape which can adhere to the cheeks of the face.

The second sheet portion 22B4 has a substantial ring shape surrounding the mouth of the face.

In such a ultrasonic probe 2A, the ultrasonic transducers 45 of the ultrasonic substrates 223 provided in each second sheet portion 22B output ultrasonic waves having relatively small opening diameters of the opening portions 411A and having frequencies equal to or greater than 2 GHz, for example. Accordingly, it is possible to avoid the risk in that an ultrasonic wave arrives at the brain.

More preferably, similar to the second embodiment, the opening diameters of the opening portions 411A of the ultrasonic transducers 45 in each ultrasonic substrate 223 are configured to be different from each other. For example, it is preferable that the first ultrasonic substrates having the ultrasonic transducers 45 outputting ultrasonic waves of 2 GHz and the second ultrasonic substrates having the ultrasonic transducers 45 outputting ultrasonic waves of 5 GHz are alternately disposed.

One ultrasonic substrate 223 may be configured to be provided with the first ultrasonic transducers outputting ultrasonic waves of 2 GHz and the second ultrasonic transducers outputting ultrasonic waves of 5 GHz.

In such a case, for example, when permeation of beauty care components or the like is input as the contents of treatment input by a user, the sequence acquisition unit 351 selects the ultrasonic transducer 45 outputting an ultrasonic wave of 2 GHz. When cleansing or a massage effect is input as the contents of treatment, the sequence acquisition unit 351 selects the ultrasonic transducer 45 outputting an ultrasonic wave of 5 GHz.

Such an ultrasonic probe 2A can perform ultrasonic treatment through a method similar to that of each embodiment. In the present embodiment, it is preferable that a gel of the fixing layer applied to the portion between the ultrasonic probe 2A and a living body (face) includes beauty care components or cleansing components. Accordingly, permeation of the beauty care components included in the gel with respect to the skin can be promoted by the ultrasonic wave, and the cleansing effect by the cleansing components can be promoted by the ultrasonic wave.

### Operational Effect of Present Embodiment

In the ultrasonic probe 2 according to the present embodiment, the plurality of second sheet portions 22B having the shapes respectively corresponding to the sites of a living body are attached to the first sheet portion 21A. In the ultrasonic probe 2 having such a configuration, for example, in a case where the shape of the treatment site is complicated, such as the face, the second sheet portions 22B are configured to have the shapes respectively corresponding to the sites. Thus, the second sheet portions 22B can be adherently fixed to a living body such that no air layer enters the space between the second sheet portions 22B and the living body.

### Modification Example of Fourth Embodiment

The above-described fourth embodiment illustrates an example of the ultrasonic probe 2A in which the dedicated second sheet portions 22B specialized for the shape of the face is attached to the first sheet portion 21A in an attachable/detachable manner. However, the second sheet portions respectively corresponding to different sites of a living body can be attached to the first sheet portion 21A.

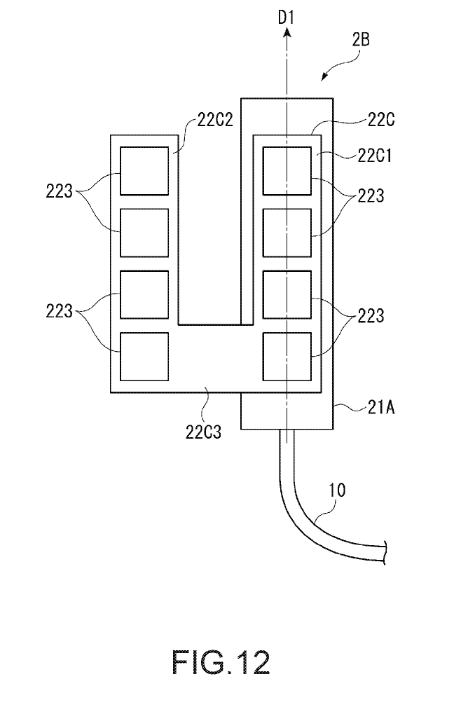

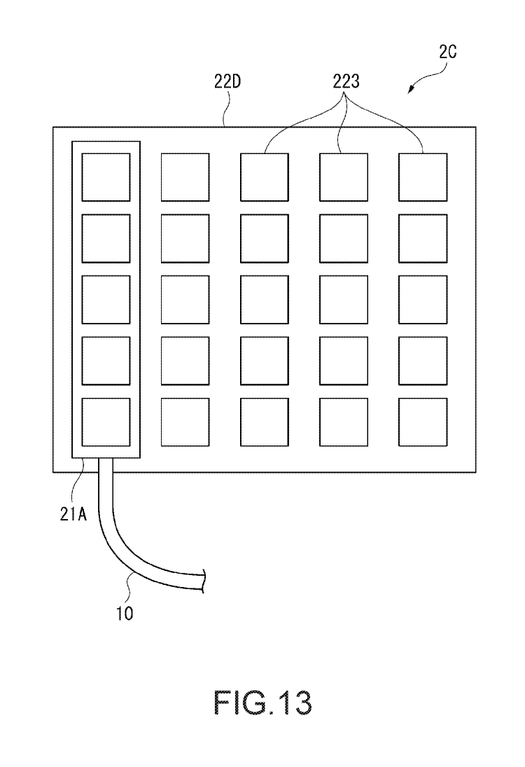

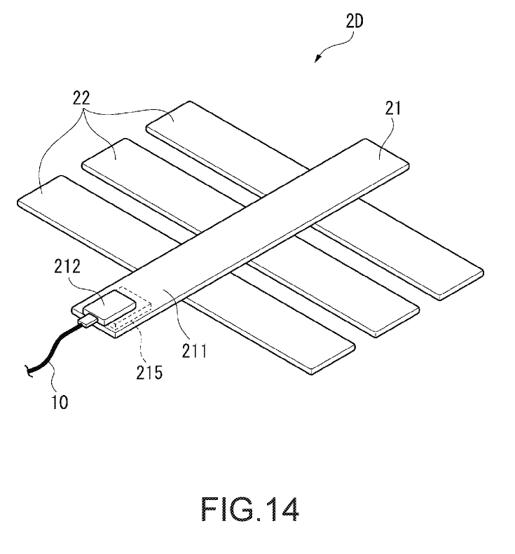

FIGS. 12 and 13 are examples of second sheet portion 22C and 22D having configurations attachable/detachable with respect to the first sheet portion 21A. FIG. 12 is a plan view illustrating an example of the shape of the second sheet portion 22C in a case where the ultrasonic probe 2A is caused to adhere to positions interposing the backbone of the back therebetween. FIG. 14 is a plan view illustrating an example of the shape of the second sheet portion 22D in a case where the ultrasonic probe 2A is caused to adhere to a relatively smooth and wide area such as the abdomen.

In an ultrasonic probe 2B illustrated in FIG. 12, the U-shaped second sheet portion 22C is fixed to the first sheet portion 21A. Specifically, the second sheet portion 22C is configured to include a first linear unit 22C1 and a second linear unit 22C2 which are parallel to the longitudinal direction (D1 direction) of the first sheet portion 21A, and an interlocking unit 22C3 which interlocks the linear units 22C1 and 22C2 at one end portion in the D1 direction, for example.

A position where the interlocking unit 22C3 is provided (a position where the linear units 22C1 and 22C2 are interlocked) is not limited to the end portion of the linear units 22C1 and 22C2. However, for example, the position may be an intermediate position in the D1 direction.

The first linear unit 22C1 is provided with a plurality of second magnets (not illustrated) respectively corresponding to the plurality of first magnets 21S and 21N (refer to FIGS. 10A and 10B) provided in the first sheet portion 21A. The first linear unit 22C1 is provided with the power receiving coils (not illustrated) respectively corresponding to the power transmitting coils 214 (refer to FIGS. 10A and 10B) of the first sheet portion 21A.

The power receiving coil may be provided at a position corresponding to any one among the plurality of power transmitting coils 214 (the power transmitting coils 214 respectively corresponding to the plurality of second sheet portions 22A in the third embodiment) provided in the first sheet portion 21A. However, a configuration provided with the plurality of power receiving coils respectively corresponding to the plurality of power transmitting coils 214 may be adopted.

In the second linear unit 22C2, a configuration provided with the plurality of magnets or the power receiving coils may be adopted.

In this case, in place of the first linear unit 22C1, the second linear unit 22C2 can be attached to the first sheet portion 21A and can be suitably changed depending on the preference of a user, the purpose of usage, and the like.

The ultrasonic probe 2B having such a configuration is caused to adhere to a living body such that a space between the first linear unit 22C1 and the second linear unit 22C2 follows along the position of the backbone. In other words, a bony portion such as the backbone reflects an ultrasonic wave. Since the backbone is located at a position close to the skin, an ultrasonic wave cannot be efficiently propagated into a living body. Therefore, it is useless even though the ultrasonic substrates 223 are disposed along the backbone and an ultrasonic wave is output. In contrast, in the above-described second sheet portion 22C, the ultrasonic probe 2B can be caused to adhere such that the ultrasonic substrates 223 are disposed at positions avoiding the backbone.

In an ultrasonic probe 2C illustrated in FIG. 13, a flat rectangular second sheet portion 22D is attached to the first sheet portion 21A.

In the second sheet portion 22D, a plurality of second magnets (not illustrated) respectively corresponding to the plurality of first magnets 21S and 21N (refer to FIGS. 10A and 10B) of the first sheet portion 21A are disposed along one end side of the rectangular shape, for example. One end side of the second sheet portion 22D is provided with the power receiving coils (not illustrated) respectively corresponding to the power transmitting coils 214 (refer to FIGS. 10A and 10B) of the first sheet portion 21A. Similar to the second sheet portion 22C illustrated in FIG. 12, a configuration provided with the plurality of power receiving coils respectively corresponding to the plurality of power transmitting coils 214 may be adopted.

The positions where the magnets or the power receiving coils are disposed are not limited to the one end side of the second sheet portion 22D. For example, the positions may be provided along the central portion.

Such a second sheet portion 22D can be caused to uniformly adhere to a skin surface where a relatively smooth area such as the abdomen can be ensured.

### MODIFICATION EXAMPLE

The invention is not limited to each of the above-described embodiments. The invention includes a configuration which can be obtained through deformation, improvement, and suitable combination of each of the embodiments within the scope in which the purpose of the invention can be realized.

For example, the above-described embodiment illustrates an example of a configuration in which the drive control circuit 226 including the pulser, the microcomputer, and the like is provided in the wiring substrate 225 of each ultrasonic substrate 223. In contrast, as illustrated in FIG. 14, a configuration in which a drive control unit for controlling driving of each ultrasonic substrate 223 is provided at one place in the ultrasonic probe 2 may be adopted. FIG. 14 is a perspective view illustrating a schematic configuration of the ultrasonic probe 2 of a different embodiment.

In an ultrasonic probe 2D illustrated in FIG. 14, a drive control circuit 215 is provided at a position where the connector portion 212 of the first sheet portion 21 is provided. The drive control circuit 215 performs drive controlling of each second sheet portion 22, each ultrasonic substrate 223, and each ultrasonic transducer group 45A. In other words, the drive control circuit 215 sequentially outputs the drive pulse to the ultrasonic transducer group 45A of the ultrasonic substrates 223 in the drive order based on the drive command data input from the control device 3.

In the third embodiment, the pulser, the microcomputer, and the like may be provided in the control circuit unit 228, and the control circuit unit 228 may perform drive controlling of each ultrasonic transducer group 45A of each ultrasonic substrate 223.

The examples illustrated in FIGS. 11 to 13 illustrate configurations in which the second sheet portions 22B, 22C, and 22D are attached to the first sheet portion 21A in an attachable/detachable manner. However, for example, a configuration of being glued and bonded to the first sheet portion 21 as those in the first and second embodiments may be adopted.

The first to third embodiments illustrate examples in which the D1 direction which is the longitudinal direction of the first sheet portions 21 and 21A is orthogonal to the D2 direction which is the longitudinal direction of the second sheet portions 22 and 22A. However, the embodiment is not limited thereto. For example, the D2 direction may be provided so as to incline at a predetermined angle with respect to the D1 direction.

The first to third embodiments illustrate examples of the second sheet portions 22 and 22A having the shapes so as to be line-symmetrical while having the first sheet portions 21 and 21A as the center. However, the embodiment is not limited thereto.

For example, a configuration in which one end portions of the second sheet portions 22 and 22A are connected to the first sheet portions 21 and 21A and extend in the direction of being separated from the first sheet portions 21 and 21A may be adopted.

The second embodiment illustrates an example in which the first ultrasonic substrates 223A outputting ultrasonic waves having the first frequencies and the second ultrasonic substrates 223B outputting ultrasonic waves having the second frequencies are alternately arranged. However, in addition thereto, a configuration in which ultrasonic waves having equal to or more than three frequencies can be output may be adopted.

The ultrasonic transducers 45 which can output equal to or more than three frequencies different from each other may be individually disposed in one ultrasonic substrate 223.

Each embodiment illustrates an example of the one-dimensional array structure in which the plurality of ultrasonic transducer groups 45A are disposed as the ultrasonic array 46 of the ultrasonic substrates 223 along the scanning direction. However, the embodiment is not limited thereto. A configuration in which each ultrasonic transducer 45 can be independently driven in the two-dimensional array structure may be adopted.

In a case of the one-dimensional array structure, scanning of an ultrasonic wave can be performed along the scanning direction (D1 direction) but scanning of an ultrasonic wave cannot be performed in the slice direction (D2 direction). In contrast, as described above, in the two-dimensional array structure, scanning of an ultrasonic wave can be performed in the D1 direction, the D2 direction, and any direction intersecting the afore-mentioned directions. Thus, it is possible to widen the selectivity of the output range of an ultrasonic wave.

The first embodiment illustrates an example in which the arrangement direction (scanning direction) of the ultrasonic transducer group 45A becomes the D1 direction which is the longitudinal direction of the first sheet portion 21. However, for example, the ultrasonic transducer groups 45A may be arranged along the D2 direction.

The first embodiment illustrates an example in which the signal lines 224 connected to the ultrasonic substrates 223 are meandering in the longitudinal direction (D2 direction) of the second sheet portions 22. In the above-described example, there is a possibility that the second sheet portions 22 are pulled in the longitudinal direction due to an operation of a user when the second sheet portions 22 are disposed, and it is possible to avoid the risk of disconnection of the signal lines 224 in such a case. Meanwhile, the meandering direction of the signal lines 224 is not limited to that described above. However, the signal lines 224 may be meandering in any direction.

The third and fourth embodiments illustrate examples in which the first sheet portion 21A and the second sheet portion 22A are fixed together by the magnets 21S and 21N, and 22S and 22N in an attachable/detachable manner. However, the embodiment is not limited thereto. For example, the first sheet portion 21A and the second sheet portion 22A may be fixed together by applying Magic Tape (registered trademark), buttoning, a double-sided tape, and the like.

An example in which power is supplied from the first sheet portion 21A to the second sheet portion 22A through the electromagnetic induction-type contactless power transmission mechanism configured to include the power transmitting coil 214 and the power receiving coil 227 is illustrated. However, the embodiment is not limited thereto. For example, a radio wave receiving-type contactless power transmission mechanism which collects power by receiving radio waves may be adopted. Otherwise, a resonance-type contactless power transmission mechanism which transmits power by causing an electric field or a magnetic field to be resonant may be adopted.

Moreover, the contactless power transmission mechanism does not have to be provided. In this case, for example, a configuration in which a power supply unit such as a secondary battery is provided in the second sheet portions 22 may be adopted. A metal pin may be provided in any one side of the first sheet portion 21A, and the second sheet portions 22A, 22B, 22C, and 22D while protruding toward the other side and the other side may have a shape including a metal terminal with which the metal pin can be engaged. In this case, power can be supplied or the drive command data can be transmitted from the first sheet portion 21A to the second sheet portions 22A, 22B, 22C, and 22D by causing the metal pin to be engaged with the metal terminal.

The above-described embodiment illustrates an example in which the ultrasonic apparatus 1 selects and sequentially drives the to-be-driven ultrasonic transducer 45 through the drive procedure based on the sequential data stored in the storage unit 34. However, for example, regardless of the treatment site, an ultrasonic wave having a uniform output value may be output. An example in which the sequential data is selected when the treatment site or the contents of treatment are input through an operation from the operation unit 31 is illustrated. However, a configuration in which, for example, an operation signal including the output value of an ultrasonic wave is input to the computation unit 35 when a user operates the operation unit 31 and the to-be-driven ultrasonic transducer 45 is selected based on the output value of the ultrasonic wave included in the operation signal may be adopted.

The above-described embodiment illustrates an example in which the ultrasonic transducers 45 output ultrasonic waves to the opening portions 411A side. However, a configuration in which ultrasonic waves are output from a side opposite to the opening portions 411A may be adopted.

An example of the piezoelectric element 413 configured to be the lamination body in which the lower electrode 414, the piezoelectric film 415, and the upper electrode 416 are laminated in the thickness direction is illustrated. However, the embodiment is not limited thereto. For example, a configuration in which a pair of electrodes are disposed on one surface side orthogonal to the thickness direction of a piezoelectric element 413 so as to face each other may be adopted. The electrodes may be disposed so as to interpose the piezoelectric film therebetween on the side surface along the thickness direction of the piezoelectric film.

Furthermore, a particular structure at the time of execution of the invention may have a configuration in which each of the above-described embodiments and the modification example are suitably combined together within the scope in which the purpose of the invention can be realized or may be suitably changed to a different structure or the like.

The entire disclosure of Japanese Patent Application No. 2015-150419 filed on Jul. 30, 2015 is expressly incorporated by reference herein.

What is claimed is:

1. An ultrasonic probe comprising:
a flexibly longitudinal first sheet portion; and
a plurality of flexible second sheet portions that are connected to one surface side of the first sheet portion,
wherein each second sheet portion has a connection surface to which the first sheet portion is connected and a drive surface on a side opposite to the connection surface and includes a plurality of ultrasonic transducers each of which can output an ultrasonic wave from the drive surface.

2. The ultrasonic probe according to claim 1,
wherein the second sheet portions are provided so as to be line-symmetrical with respect to the first sheet portion.

3. The ultrasonic probe according to claim 1,
wherein the second sheet portions are longitudinal in a direction intersecting a longitudinal direction of the first sheet portion.

4. The ultrasonic probe according to claim 1,
wherein each second sheet portion has a longitudinal direction, and
wherein each second sheet portion is provided with wiring which is connected to the ultrasonic transducers and is meandering in the longitudinal direction of the second sheet portion

5. The ultrasonic probe according to claim 1,
wherein each second sheet portion has wiring which is connected to the ultrasonic transducers, and
wherein the wiring connects the plurality of ultrasonic transducers together in parallel.

6. The ultrasonic probe according to claim 1,
wherein each ultrasonic transducer includes a first ultrasonic transducer which can output an ultrasonic wave having a first frequency and a second ultrasonic transducer which can output an ultrasonic wave having a second frequency different from the first frequency.

7. The ultrasonic probe according to claim 1,
wherein the second sheet portions are provided so as to be attachable/detachable with respect to the first sheet portion.

8. The ultrasonic probe according to claim 7,
wherein the first sheet portion includes first magnets at positions where the first sheet portion is connected to the second sheet portions, and
wherein each second sheet portion includes a second magnet having magnetism opposite to that of the first magnet and is connected to the first sheet portion due to magnetic force.

9. The ultrasonic probe according to claim 7,
wherein the first sheet portion has power transmitting coils at positions where the first sheet portion is connected to the second sheet portions, and
wherein each second sheet portion has a power receiving coil at a position where the second sheet portion is connected to the first sheet portion, and power is supplied from the power transmitting coils to each power receiving coil through contactless power transmission.

10. An ultrasonic apparatus comprising:
the ultrasonic probe according to claim 1; and
a control unit that controls the ultrasonic probe.

11. An ultrasonic apparatus comprising:
the ultrasonic probe according to claim 2; and
a control unit that controls the ultrasonic probe.

12. An ultrasonic apparatus comprising:
the ultrasonic probe according to claim 3; and
a control unit that controls the ultrasonic probe.

13. An ultrasonic apparatus comprising:
the ultrasonic probe according to claim 4; and
a control unit that controls the ultrasonic probe.

14. An ultrasonic apparatus comprising:
the ultrasonic probe according to claim 5; and
a control unit that controls the ultrasonic probe.

15. An ultrasonic apparatus comprising:
the ultrasonic probe according to claim 6; and
a control unit that controls the ultrasonic probe.

16. An ultrasonic apparatus comprising:
the ultrasonic probe according to claim 7; and
a control unit that controls the ultrasonic probe.

17. An ultrasonic apparatus comprising:
the ultrasonic probe according to claim 8; and
a control unit that controls the ultrasonic probe.

18. The ultrasonic apparatus according to claim 10, further comprising:
a storage unit that stores a target subject which is an output target of an ultrasonic wave and sequential data which indicates an output procedure of an ultrasonic wave with respect to the target subject,
wherein the control unit causes the plurality of ultrasonic transducers to respectively output ultrasonic waves, based on the sequential data.

19. The ultrasonic apparatus according to claim 18,
wherein the sequential data includes drive element data which indicates the position of a to-be-driven ultrasonic transducer among the plurality of ultrasonic transducers, and
wherein the control unit selects the to-be-driven ultrasonic transducer from the plurality of ultrasonic transducers based on the drive element data and drives the selected ultrasonic transducer.

20. The ultrasonic apparatus according to claim 18,
wherein the sequential data includes output range data which indicates an output range of an ultrasonic wave, and
wherein the control unit performs delay-driving of the plurality of ultrasonic transducers based on the output range data.
